(19) United States
(12) Patent Application Publication
(10) Pub. No.: US 2006/0217731 A1
(43) Pub. Date: Sep. 28, 2006

(54) X-ray and fluoroscopic visualization slots

(75) Inventors: Carlos Gil, Collierville, TN (US); Loic Josse, Paris (FR)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/091,060

(22) Filed: Mar. 28, 2005

(51) Int. Cl. A61F 5/00

(52) U.S. Cl. 606/86

(57) ABSTRACT

A method of preparing an intervertebral disc space between a pair of vertebral bodies to receive an implant comprises engaging a portion of a surgical instrument with at least one of the vertebral bodies. The surgical instrument has a passage therethrough. The method further comprises generating one or more images of the surgical instrument in the surgical field and visualizing the passage through the surgical instrument on the one or more images. The method further comprises aligning the surgical instrument with a surgical plane through the intervertebral disc space by maintaining visualization of the through passage on the one or more images while adjusting the surgical instrument.

### BACKGROUND

Technical advances in the design of joint reconstructive devices have revolutionized the treatment of degenerative joint disease, moving the standard of care from arthrodesis to arthroplasty. Reconstruction of a damaged joint with a functional joint prosthesis to provide motion and to reduce deterioration of the adjacent bone and adjacent joints is a desirable treatment option for many patients. For the surgeon performing the joint reconstruction, specialized instrumentation and surgical methods, particularly improved instrument alignment tools, may be useful to facilitate precise placement of the prosthesis.

### SUMMARY

In one embodiment, a method of preparing an intervertebral disc space between a pair of vertebral bodies to receive an implant comprises engaging a portion of a surgical instrument with at least one of the vertebral bodies. The surgical instrument has a passage therethrough. The method further comprises generating one or more images of the surgical instrument in the surgical field and visualizing the passage through the surgical instrument on the one or more images. The method further comprises aligning the surgical instrument with a surgical plane through the intervertebral disc space by maintaining visualization of the through passage on the one or more images while adjusting the surgical instrument.

In another embodiment, a surgical instrument comprises an instrument body at least a portion of which is adapted for interposition between a pair of vertebral bodies and a first through opening in the instrument body. The first through opening defining a first axis through the instrument body. The first through opening is adapted for visualization with an imaging device when the first axis is generally perpendicular to a first image plane generated by the imaging device.

In another embodiment, a method for assessing an orientation of a surgical instrument in a surgical field comprises positioning at least a portion of the surgical instrument in the surgical field. The surgical instrument has first and second passages therethrough. The method further comprises generating a first image of the surgical instrument, visualizing an axis of sight through the first passage in the surgical instrument on the first image, and generating a second image of the surgical instrument. The method further comprises visualizing an axis of sight through the second passage in the surgical instrument on the second image.

### BRIEF DESCRIPTION OF THE DRAWINGS

FIG. 1 is a side view of a vertebral column having a damaged disc.

FIG. 2 is a perspective view of an alignment guide according to an embodiment of the current disclosure.

FIG. 3 is a perspective view of an anchoring device according to an embodiment of the current disclosure.

FIG. 4 is an environmental view of the assembled alignment guide and anchoring device of FIGS. 2 & 3.

FIG. 5 is a cross sectional view of a surgical instrument according to another embodiment of the present disclosure.

FIG. 6 is an environmental view of the surgical instrument of FIG. 5.

FIG. 7 is a perspective view of an implantable prosthesis according to another embodiment of the present disclosure.

### DETAILED DESCRIPTION

The present disclosure relates generally to the field of orthopedic surgery, and more particularly to instrumentation and methods for spinal surgery. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

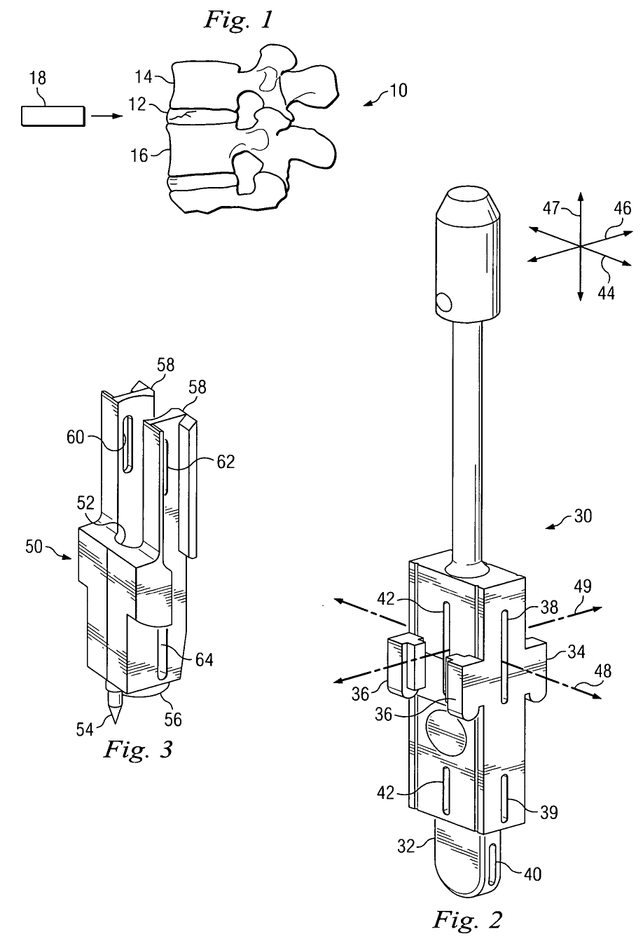

Referring first to FIG. 1, the numeral 10 refers to a human anatomy having a joint location which, in this example, includes an injured, diseased, or otherwise damaged intervertebral disc 12 extending between vertebrae 14, 16. The damaged disc may be replaced by an intervertebral disc prosthesis 18 which may be a variety of devices including any of the prostheses which have been described in U.S. Pat. Nos. 5,674,296; 5,865,846; 6,156,067; 6,001,130; 6,740,118 and in U.S. Patent Application Pub. Nos. 2002/0035400; 2002/0128715; and 2003/0135277; 2004/0225366 which are incorporated by reference herein.

A variety of surgical techniques may be used to repair the damaged joint. First, all or a portion of the damaged tissue including disc 12 may be excised. This procedure may be performed using an anterior, anterolateral, lateral, or other approach known to one skilled in the art, however, the following embodiments will be directed toward a generally anterior approach unless otherwise specified. Generally, the tissue removal procedure may include positioning and stabilizing the patient. Fluoroscopic, x-ray and/or other imaging methods may be used to assist with vertebral alignment and surgical guidance. The tissue surrounding the disc space may be retracted to access and verify the target disc space. Imaging techniques may also be used to determine the proper sizing of the intervertebral prosthesis 18. In one embodiment, a sizing template may be used to pre-operatively determine the correct prosthesis size. The area of the target disc may be prepared by removing excess bone, including osteophytes which may have developed, and other tissues which may include portions of the annulus and all or portions of the nucleus pulpous. The tissue removal procedure, which may include a discectomy procedure, may alternatively or additionally be performed after alignment and/or measurement procedures have been taken.

Certain surgical instruments that will be described below are similar to the instrumentation described in U.S. patent application Ser. No. 10/799,178 (“the '178 application”) entitled “Technique and Instrumentation for Intervertebral Prosthesis Implantation Using Independent Landmarks,” filed Mar. 12, 2004 which is incorporated by reference herein.

Various alignment procedures may be conducted with surgical instrumentation to align the intervertebral space in preparation for the disc prosthesis 18. The transverse center of the disc space may be determined and marked. Referring now to FIG. 2, in one embodiment, a surgical instrument used for alignment may be an alignment guide 30, comprising an intervertebral portion 32. The intervertebral portion 32 may be selected to permit insertion between the adjacent vertebrae 14, 16 with minimal distraction. The alignment guide 30 may further comprise positioning guides 34, 36. In one embodiment, as illustrated in FIG. 2, the positioning guides 34, 36 may have differing lengths to facilitate easy coupling to subsequent instrumentation.

Slots 38, 39, 40, 42 may extend through the body of the alignment guide 30, providing a radiolucent pathway visible when imaged with imaging equipment such as x-ray or fluoroscopic equipment. These slots, passages, or openings 38, 40, 42 may allow visualization through the guide 30, thereby allowing a user to view a generated image to determine whether the alignment guide is oriented properly. On the generated image, the through slots 38, 40, 42 may appear as a contrasting shade. The slots 38 and 40 may have a generally narrow, elongated shape and may extend in a direction 44 which may be a transverse axis when the alignment guide 30 is in use. The slots 42 may also have a generally narrow, elongated shape and may extend in a direction 46 which may be a sagittal axis when the alignment guide 30 is in use. Slot 38 may have a through axis of sight 48, and slot 42 may have a through axis of sight 49.

Referring now to FIG. 3, an anchoring device 50 may include a vertebral body attachment portion 52, a restraint pin 54, a seat.56, and constraint members 58. The restraint pin 54 may be retractable or fixed. The anchoring device 50 may further include slots 60, 62, 64. These slots may allow visualization through the device 50 when observed with imaging equipment such as x-ray or fluoroscopic equipment. The slots 58, 60, 62 may have a generally narrow, elongated shape and may extend in the direction 44 when the anchoring device 50 is in use.

The shape and location of the slots 38, 40, 42, 58, 60, 62 are merely exemplary and it is understood that in alternative embodiments, the slots may have a circular cross-section, a square cross-section or any other shape. The slots may be empty as shown in the embodiments of FIGS. 2 and 3, however the slots may also be filled or plugged with a radiolucent material or other material that provides contrast when viewing the slot on a generated image.

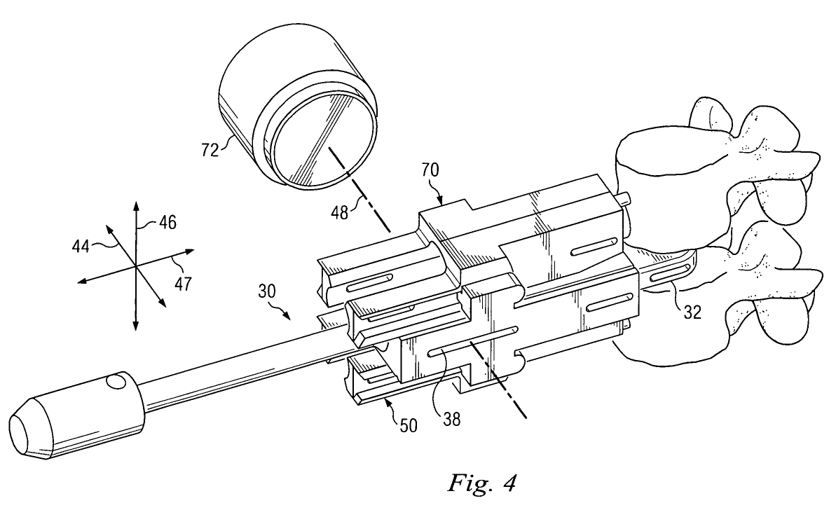

The anchoring device 50 may attach to surgical instrumentation such as the distracting assembly described in the '178 application. Alternatively, as shown in FIG. 4, the anchoring device 50 may attach to the alignment guide 30. Specifically, in the illustrated embodiment, one set of positioning guides, for example guides 36, may mate with the constraint portions 58. The constraint portions 58 may prevent movement of the alignment guide 30 relative to the anchoring device 50 respectively. The alignment guide 30 may also engage with the anchoring device 50 by magnetic or other mechanical connections. As shown in FIG. 4, the alignment guide may be connected to a second anchoring device 70. The anchoring device 70 may be substantially similar to anchoring device 50 and therefore will not be described in detail.

With the alignment guide 30 coupled to the anchoring devices 50, 70 the intervertebral portion 32 may be inserted between the vertebral endplates of vertebral bodies 14, 16. Alternatively, the insertion of intervertebral portion 32 between the vertebral endplates may take place before or as the alignment guide 30 is coupled to the anchoring devices 50, 70. The alignment guide 30 and the anchoring devices 50, 70 may be aligned relative to the vertebral bodies 14, 16 using the imaging equipment 72, which may be x-ray or fluoroscopic equipment, and the slots 38, 39, 40, 42, 60, 62, 64. For example, sagittal alignment of the guide 30 may be accomplished by positioning an imaging device 72 such that the image plane is parallel to a sagittal plane through the surgical field. The position of the alignment guide 30 may be adjusted until the generated image fully shows the open slot 38. In this position, the axis of sight 48 through the slot 38 may be generally perpendicular to the plane of the image. Further, transverse alignment of the alignment guide 30 may be accomplished by positioning an imaging device (which may be the same or different than imaging device 72) such that the image plane is parallel to a transverse plane through the surgical field. The position of the alignment guide 30 may be adjusted until the generated image fully shows the open slot 42. In this position, the axis of sight 49 through the slot 42 may be generally perpendicular to the plane of the image. The slots 39, 40 may also be used to orient the alignment guide 30 using a process similar to that described above. Likewise, the slots 60, 62, and 64 may be used to orient the anchoring device 50 relative to the vertebral bodies 14, 16 using a process similar to that described above.

In an alternative embodiment, if the alignment guide is to be angled with respect to the image plane, the slot may be formed at an angle through the alignment guide such that an axis of sight through the slot is still perpendicular to the image plane. In this way the axis of sight through the slot may be visualized on the generated image. In this embodiment, an opening of the slot may be in a plane that intersects the plane of the image.

With the alignment verified, a hole may be drilled into the caudal vertebral body 16 through the vertebral body attachment portion 52 of the anchoring device 50. An anchoring fixture, such as a bone screw (not shown), may be inserted through the vertebral body attachment portion 52 and into the vertebral body 16 thus firmly locking the seat 56 to the vertebral body 16. As the bone screw descends through the vertebral body attachment portion 52, the restraint pin 54 may become embedded in the vertebral body 16 to prevent rotation of the anchoring device 50 and the subsequent loosening of the anchoring device 50 from the vertebral body 16.

The seat 56 of the anchoring device 50 may be adjustable and thus may be raised, lowered, and/or tilted. With the seat 56 engaged with the vertebral body 16, the corresponding seat of the cephalad anchoring device 70 may be adjusted to contact the vertebral body 14, maintaining the alignment guide 30 aligned in a generally anterior-posterior direction. With the seat of anchoring device 70 in position, a second hole may be drilled into the cephalad vertebral body 14 through the vertebral body attachment portion of the anchoring device 70. Another anchoring fixture, such as a bone screw, may be inserted through the anchoring device 70 and into the vertebral body 14 thus firmly locking the anchoring device to the vertebral body 14. A restraint pin similar to pin 54 may then deploy into vertebral body 14. The anchoring devices 50, 70 may become directly engaged with the vertebral bodies, and the alignment device may become directly or indirectly engaged with the vertebral bodies. It is understood that in an alternative embodiment, the cephalad anchoring devices 70 may be placed before the caudal anchoring device 50. With the anchoring devices 50, 70 in place, the alignment guide 30 may be removed. The anchoring devices 50, 70 may remain in place to anchor additional instruments such as disc space milling tools, distraction instruments, and/or implant insertion tools.

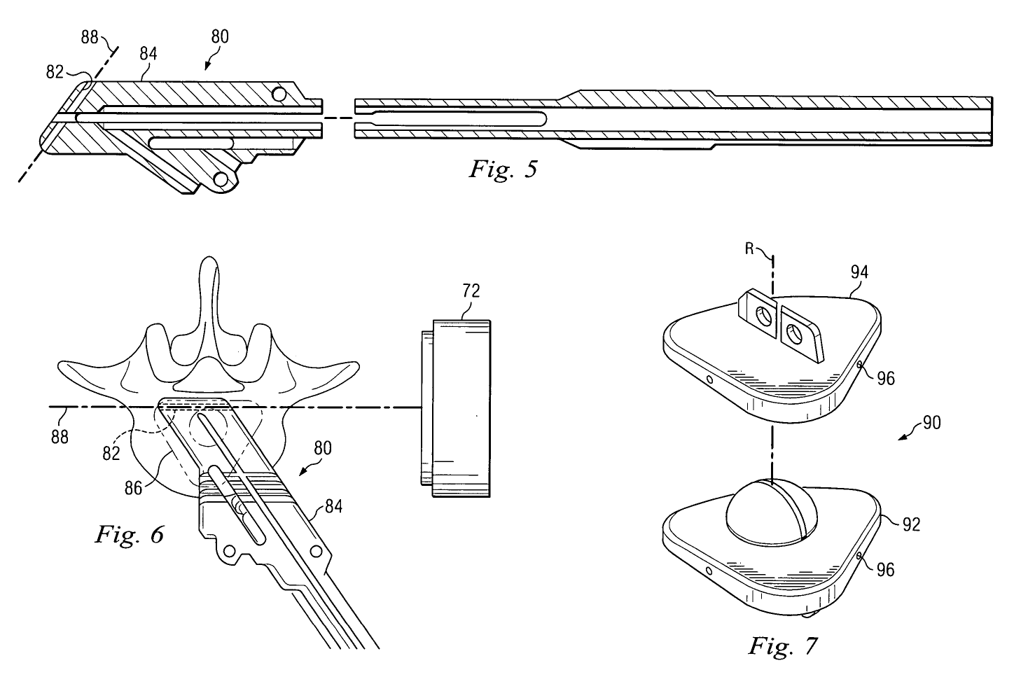

Referring now to FIGS. 5 and 6, an implant insertion guide 80 may have a slot 82 through a distal end 84. The insertion guide 80 may be used to implant a disc prothesis 86 between the vertebral bodies 14, 16. The slot 82 may provide a radiolucent pathway visible when imaged with imaging equipment such as x-ray or fluoroscopic equipment. The slot 82 may permit visualization through the guide 80, thereby allowing a user to view a generated image to determine whether the guide 80 is oriented properly. The slot 82 may have a generally narrow, elongated shape and may have a through axis of sight 88.

The insertion guide 80 may be used to guide the prosthesis 86 into the intervertebral disc space using an anterior-oblique approach. The slot 82 may be visible on an image generated by the imaging device 72 when the axis of sight 88 is positioned generally perpendicular to the plane of the generated image. The slot 82 may be used to orient both the sagittal and anterior-posterior placement of the guide 80 and consequently the prosthesis 86 within the intervertebral disc space.

Referring now to FIG. 7, alignment slots such as those described above may be used with implantable instrumentation and devices. For example an intervertebral articulating prosthetic joint 90, similar to that described in U.S. patent application Ser. No. 10/774,157 entitled “Articular Disc Prosthesis for Anterior-Oblique Insertion” and incorporated by reference herein, may have a first articular component 92 and a second articular component 94. The articular components 92, 94 cooperate to form the prosthetic joint 90 which is sized and configured for disposition within an intervertebral space between the adjacent vertebral bodies 14, 16. The articular components 92, 94 may have through slots 96 which may be angled such that they are visible with imaging equipment and may be used to assess the intraoperative orientation of the joint 90.

In alternative embodiments, other types of surgical instrumentation may be aligned using alignment slots such as those described above. Such instrumentation may include distraction equipment, cutting tools, trial devices, insertion tools, revision tools, and diagnostic tools. Additionally, alignment slots may be used with implantable instrumentation such as vertebral fusion devices or any design of motion preservation implant. In still another alternative embodiment, the alignment slots may be used with instrumentation in other non-spinal surgical procedures. The alignment slots may be used with both orthopedic instrumentation or non-orthopedic instrumentation whenever alignment using imaging techniques such as x-ray or fluoroscope is possible.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “cephalad,” and “caudal,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A method of preparing an intervertebral disc space between a pair of vertebral bodies to receive an implant, the method comprising:
engaging a portion of a surgical instrument with at least one of the vertebral bodies, the surgical instrument having a passage therethrough;
generating one or more images of the surgical instrument in the surgical field;
on the one or more images, visualizing the passage through the surgical instrument; and
aligning the surgical instrument with a surgical plane through the intervertebral disc space by maintaining visualization of the through passage on the one or more images while adjusting the surgical instrument.

2. The method of claim 1 further comprising inserting a portion of the surgical instrument between the pair of vertebral bodies.

3. The method of claim 1 wherein the passage is an elongated slot through the surgical instrument.

4. The method of claim 1 wherein the passage has a generally circular cross section.

5. The method of claim 1 wherein the surgical plane is a sagittal plane through the intervertebral disc space.

6. The method of claim 1 wherein the surgical plane is a transverse plane through the intervertebral disc space.

7. The method of claim 1 wherein an opening of the passage is in a plane that intersects a plane of the one or more images.

8. A surgical instrument comprising:
an instrument body at least a portion of which is adapted for interposition between a pair of vertebral bodies and
a first through opening in the instrument body, the first through opening defining a first axis through the instrument body,
wherein the first through opening is adapted for visualization with an imaging device when the first axis is generally perpendicular to a first image plane generated by the imaging device.

9. The surgical instrument of claim 8 wherein the instrument body is an alignment guide for aligning an intervertebral disc space between the pair of vertebral bodies to receive a prosthesis.

10. The surgical instrument of claim 8 wherein the instrument body is a device for coupling to at least one of the vertebral bodies.

11. The surgical instrument of claim 8 wherein the instrument body is a prosthesis for implantation between the pair of vertebral bodies.

12. The surgical instrument of claim 8 wherein the instrument body is a prosthesis insertion guide.

13. The surgical instrument of claim 8 wherein the first through opening is an elongated slot.

14. The surgical instrument of claim 8 wherein the first through opening is a hole having a generally circular cross-section.

15. The surgical instrument of claim 8 wherein the imaging device is x-ray equipment.

16. The surgical instrument of claim 8 wherein the imaging device is fluoroscopic equipment.

17. The surgical instrument of claim 8 further comprising:
a second through opening in the instrument body, the second through opening defining a second axis through the instrument body,
wherein the second through opening is visible with the imaging device and the second axis is perpendicular to the first image plane generated by the imaging device.

18. The surgical instrument of claim 8 further comprising:
a second through opening in the instrument body, the second through opening defining a second axis through the instrument body,
wherein the second through opening is visible with the imaging device and the second axis is perpendicular to a second image plane generated by the imaging device and
wherein the first axis and the second axis are skew or intersecting.

19. A method for assessing an orientation of a surgical instrument in a surgical field comprising:
positioning at least a portion of the surgical instrument in the surgical field, the surgical instrument having first and second passages therethrough;
generating a first image of the surgical instrument;
on the first image, visualizing an axis of sight through the first passage in the surgical instrument;
generating a second image of the surgical instrument; and
on the second image, visualizing an axis of sight through the second passage in the surgical instrument.

20. The method of claim 19 wherein a plane of the first image intersects a plane of the second image.

21. The method of claim 19 wherein the axis of sight through the first passage is perpendicular to a sagittal plane through the surgical field.

22. The method of claim 19 wherein the axis of sight through the second passage is perpendicular to a transverse plane through the surgical field.

23. The method of claim 19 wherein the axis of sight through the first passage is perpendicular to a plane of the first image.
